(19) United States
(12) Patent Application Publication
(10) Pub. No.: US 2013/0096413 A1
(43) Pub. Date: Apr. 18, 2013

(54) OPTICAL ULTRASOUND TRANSDUCER

(71) Applicant: Regents of the University of Minnesota, St. Paul, MN (US)

(72) Inventors: Shai Ashkenazi, St. Louis Park, MN (US); Clay Smith Sheaff, Minneapolis, MN (US)

(73) Assignee: REGENTS OF THE UNIVERSITY OF MINNESOTA, St. Paul, MN (US)

(21) Appl. No.: 13/651,063

(22) Filed: Oct. 12, 2012

Related U.S. Application Data
(60) Provisional application No. 61/546,309, filed on Oct. 12, 2011.

(51) Int. Cl. A61B 6/00; B23P 17/00

(52) U.S. Cl. 600/407; 29/527.2

(57) ABSTRACT

In general, this disclosure describes various optical ultrasound transducers and methods of producing such. As one example, an optical ultrasound transducer comprises an optical fiber and a polymer layer formed on the optical fiber to receive light from the optical fiber. The polymer layer may absorb light of a first wavelength and be substantially transparent to light of a second wavelength. In response to the light of the first wavelength, the polymer layer may generate an acoustic tone. The optical ultrasound transducer may further include an optical detector formed on the polymer layer, the optical detector comprising an etalon structure having a first mirror layer and a second mirror layer separated by a compressible layer, wherein the compressible layer resonates in response to the light of the second wavelength passing through the polymer layer and is compressible in response to acoustic pressure from echoes of the acoustic tone.

This application claims the benefit of application number Ser. No. 61/546,309, filed Oct. 12, 2011, the entire content of which is incorporated herein by reference.

### TECHNICAL FIELD

The invention relates to imaging devices and, more particularly, to ultrasound imaging devices.

### BACKGROUND

High-frequency ultrasound (HFUS) has been used to generate high-resolution (<100 μan) images in medical applications such as endoscopy, intravascular imaging, ophthalmology, and dermatology. The production of HFUS transducers, however, has proven to be difficult using conventional design and manufacturing techniques. Thin-film PVDF and capacitive transducers (CMUT) have circumvented the difficulties in dicing piezoceramics on the micron scale, however the electrical connections required still make these devices susceptible to excessive noise due to crosstalk, RF interference, and small capacitance. These factors severely limit image quality.

### SUMMARY

Devices that optically generate and detect ultrasound circumvent the problems intrinsic to small-scale piezoelectric transducers by requiring no electrical cabling or interconnections. An etalon in as an optical device containing parallel, partially-reflective mirrors. Thin-film etalons are good candidates for optical ultrasound sensor arrays and exhibit the high sensitivity and large bandwidth required for high-resolution imaging. They are also relatively easy to manufacture using nanofabrication techniques. These devices operate by subjecting a small and compressible Fabry-Pérot interferometer to high-frequency ultrasound (HFUS) which in turn modulates the optical cavity thickness. This change in thickness alters the optical path length thereby resulting in a shift in the resonance wavelength. If the probe beam's wavelength is tuned on either edge of the resonance, a corresponding change in the beam's reflected intensity occurs and can be captured using a photo detector. A distinct advantage of etalon sensors is that the sensitivity does not decrease as the active area is decreased. Furthermore the active area of the sensing element is merely dependent on the spot size of the probe beam. The size of the element can therefore be easily reduced to a spot of diameter less than 100 μm by using a focusing lens. This generates a point source-like detector which provides for a wide acceptance angle.

HFUS can also be generated via the photoacoustic effect—the conversion of optical energy into a thermoelastic wave. While the most common method of photoacoustic excitation in medical imaging is the direct irradiation of tissue, photoabsorptive thin films can be used as photoacoustic targets for use in pulse-echo mode. Moreover, the simple nature of these films allows them to be integrated into etalon structures so as to provide an all-optical transmit/receive ultrasound sensor. A transducer may be created by transforming one of the etalon mirrors into a periodic gold nanostructure. When exposed to a short laser pulse at the structure's plasmon resonance frequency, a thermoelestic wave is generated. By designing the dimensions of the nanostructure appropriately, this resonance frequency occurs sufficiently distal to that of the etalon structure thereby allowing dual-mode functionality with the use of two optical sources. However, this structure is difficult to fabricate and unfortunately has a low damage threshold which makes long-term use unviable. A photoabsorptive black polydimethylsiloxane (PDMS) layer may be introduced on top of an unmodified etalon, however this configuration introduces two significant disadvantages: (1) it requires the transmitting and sensing elements to be in different locations which was shown to reduce bandwidth and hinder image reconstruction, and (2) deposition of the transmitting layer on top of the etalon introduces acoustic attenuation and decreases the sensor's bandwidth by effectively making the device thicker.

An all-optical ultrasound transducer is described herein that integrates an optically-absorbing polyimide thin-film into an etalon sensor. This optical technique provides for very small ultrasound transducers. Transmission and reception of the ultrasound is based upon optical interfaces. A laser is delivered to the device optically, which is absorbed and causes the device to emit ultrasound. A second layer acts as a resonator and is sensitive to pressure of the ultrasound and therefore provides a way to detect the ultrasound echoes. The device forms a transmitter receiver for ultrasound while the interface to the outside world is through optical signals and not electronic signals.

One advantage of opto-acoustic technology that is based on optic signaling is that the element described herein can be made very small depending upon the optics. For example, the device could focus down to 10 micron, so the effective area of a transducer in accordance with the techniques herein could be 10 micron in this example. The opto-acoustic technology may be applied for miniaturized imaging probes, as one example, including ultrasound imaging probes. Example applications include intravascular imaging, intracardiac or any image guided interventions, such as laparoscopic surgeries, where visual feedback is needed. In these applications, imaging probes in accordance with the techniques described herein may be less invasive than conventional techniques because of the reduced size of the transducers. Moreover, more intense light rays may be utilized so as to provide improved imaging sensitivity. The number of imaging elements may also be increased due to the reduced size, which may aid in forming higher quality of images.

The optical and acoustic properties of the device as well as the imaging capabilities of the device are described herein. An example device design for high resolution imaging applications is described. Because the opto-acoustic transduction mechanisms rely on light delivery, the coupling of a 2-D transmit/receive array with optical fibers provides a compact and flexible device well suited for endoscopic and intravascular ultrasound (IVUS).

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an example optical ultrasound transducer, in accordance with one or more aspects of the present disclosure.

FIG. 2 is a block diagram illustrating an example optical ultrasound transducer, in accordance with one or more aspects of the present disclosure.

FIG. 3 is a block diagram illustrating an example optical ultrasound transducer, in accordance with one or more aspects of the present disclosure.

FIG. 4 is a block diagram illustrating an example optical ultrasound transducer, in accordance with one or more aspects of the present disclosure.

FIG. 5 is a block diagram illustrating an example optical ultrasound transducer, in accordance with one or more aspects of the present disclosure.

FIG. 6 is a block diagram illustrating an example layered microstructure and operating principle of an all-optical, thin-film, high-frequency ultrasound transducer, in accordance with one or more aspects of the present disclosure.

FIG. 7 is a graphical diagram showing an example resonance profile of an etalon sensor when optically tested, in accordance with one or more aspects of the present disclosure.

FIGS. 8A and 8B are graphical diagrams showing example pulse-echoes from an optical ultrasound transducer, in accordance with one or more aspects of the present disclosure.

FIGS. 9A and 9B are graphical diagrams showing a recorded waveform and associated frequency response of an optical ultrasound transducer, in accordance with one or more aspects of the present disclosure.

FIGS. 10A-10C are graphical diagrams showing etalon detection of a polyimide pulse-echo in an optical ultrasound transducer, in accordance with one or more aspects of the present disclosure.

FIG. 11A and 11B are a block diagram and associated graphical diagram illustrating an example 1-D synthetic aperture scanning system, in accordance with one or more aspects of the present disclosure.

FIG. 12 is a block diagram illustrating an example configuration of an optical ultrasound transducer, in accordance with one or more aspects of the present disclosure.

### DETAILED DESCRIPTION

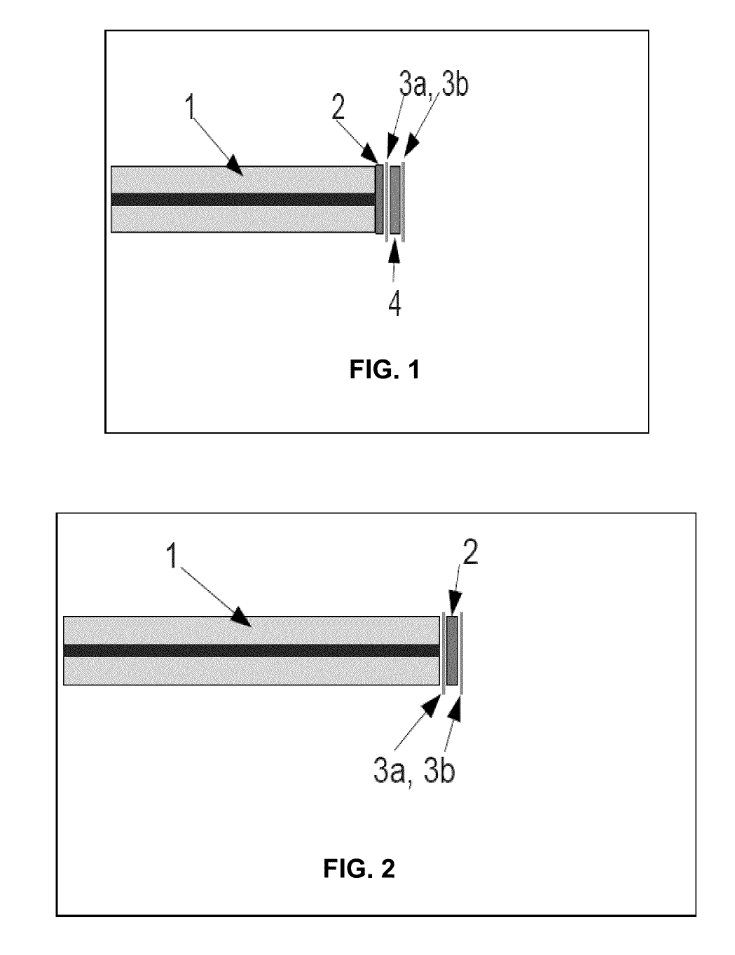

FIG. 1 is a block diagram illustrating an example optical ultrasound transducer, in accordance with one or more aspects of the present disclosure. In the example shown in FIG. 1, an ultrasound transducer includes single mode fiber optic (SMF) 1, coated with high optical absorption thermoelastic material layer 2 (hereinafter “PI layer 2”), typically 1-2 μm in width. In other examples, SMF 1 may instead be a multimode fiber optic (MMF). In the example of FIG. 1, SMF 1 is also coated with a first reflecting surface, layer 3a, a transparent polymer, layer 4, and a second reflecting surface, layer 3b. A laser pulse (e.g., of UV light) at a wavelength within the absorbing range of PI layer 2 (e.g. 355 nm) is delivered through SMF 1 and is absorbed by PI layer 2. PI layer 2 acts as a transmitter that absorbs light at specific wavelengths in order to generate an acoustic tone. PI layer 2 is transparent to other wavelengths, such that light can propagate through PI layer 2 and probe the detector, which comprises layers 3a, 4, and 3b. For example, PI layer 2 may comprise a material that has very high absorption in the ultra-violet (UV) light range and very good transmission characteristics in the infra-red light range. PI layer 2 may be a polymer or a mixture of a dye and a polymer that has high optical absorption at a specified range (absorption range) and high optical transmission at a different wavelength range (transmission range). As an example, PI can stand for Polylmide polymer. In this case the absorption range would include 200 nm<λ<400 nm, and the transmission range would include 600 nm<λ<2000 nm.

In the detector (i.e., layers 3a, 4, 3b), light transmits back and forth between the two mirrors of layer 3a and 3b, and any pressure applied to one of the mirrors creates change in the directivity. The pulse absorption thereby generates ultrasound waves by the thermoelastic mechanism. A second laser (e.g., one having a continuous wavelength at 1550 nm) is delivered through SMF 1 and is used to probe the etalon structure of layers 3a, 4, 3b. Layer 4 is a compressible polymer layer which acts as a spacer between the two reflecting surfaces 3a and 3b. Because layer 4 is compressible, it is responsive to acoustic pressure and the distance between the two reflecting mirrors (i.e., layers 3a, 3b) is modified by the acoustic wave. The etalon structure of layers 3a, 4, 3b allows a specific wavelength or a specific wavelength range to penetrate into the space between the mirrors and resonate back. A resonance shift occurs in response to compression of layer 4. This change in the distance is probed by the continuous wavelength laser. The reflection of the second laser is measured by a photodetector (PD). The PD output is converted from current to voltage and then sampled by an analog-to-digital converter (A/D). The digital signal received from the A/D converter corresponds to the acoustic pressure at the active area of the device (i.e., the tip of SMF 1).

FIG. 2 is a block diagram illustrating another example optical ultrasound transducer, in accordance with one or more aspects of the present disclosure. In the example of FIG. 2, SMF 1 is coated with a first reflecting surface, layer 3a, PI layer 2, and a second reflecting surface, layer 3b. PI layer 2 may typically be, in this example, 5-10 μm in width. Layer 3a may be a wavelength-selective mirror coating design to transmit short waves (including uv) and reflect long waves (including near infra-red 1550 nm). As shown, PI layer 2 is formed between layers 3a and 3b and operates both as a compressible spacer (receiver) and a pulse converter (transmitter). PI layer 2 is selected to have very good transmission. That is, PI layer 2 may be relatively transparent in the near infra-red (NIR), allowing it to act as the spacer between the mirrors of layers 3a, 3b, but at the same time it may have good absorption for UV light and thereby operate as a pulse converter to convert the UV light to ultrasound as in the configuration of FIG. 1.

In the example of FIG. 2, a laser pulse at a wavelength within the absorbing range of PI layer 2 (e.g. 355 nm) is delivered through SMF 1 and is absorbed by PI layer 2. In this example, layer 3a is transparent to UV such that the UV pulse is transmitted into PI layer 2 and absorbed to generate the ultrasound. The pulse absorption generates ultrasound waves by the thermoelastic mechanism. A second laser, such as a continuous wave (CW) laser at 1550 nm, is delivered through SMF 1, and is used to probe the etalon structure of layers 3a, 2, 3b. The reflection of the CW laser is measured by a PD. The PD output is converted from current to voltage and then sampled by an A/D converter. The digital signal corresponds to the acoustic pressure at the active area of the device (i.e., the tip of SMF 1).

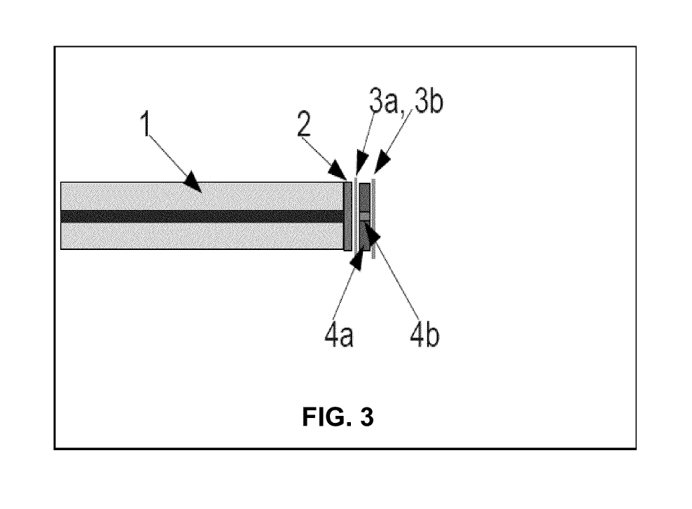

FIG. 3 is a block diagram illustrating an example optical ultrasound transducer, in accordance with one or more aspects of the present disclosure. As shown, SMF 1 is coated with PI layer 2 and a first reflecting surface, layer 3a. As shown in FIG. 3, a layer of transparent polymer, layer 4b, is then coated on layer 3a. Layer 4b is then patterned to remove material not in front of the SMF core of SMF 1. A second polymer layer, layer 4a, is then applied to fill the space of the removed material. A second reflecting surface, layer 3b, is then coated on to form the detector. The optical refraction index of layer 4b is higher than the refraction index of layer 4a. This reduces any lateral divergence of the light from the detector as the light emerges from the optical fiber, SMF 1, thereby reducing any loss of energy from the resonator. As a result, the device may achieve higher quality factor (Q-factor) of its optical resonance and therefore higher acoustic sensitivity. In a sense, layers 4a, 4b operate to extend the fiber cladding and fiber core of SMF 1, correspondingly, into the etalon structure of layers 3a, 4a, 4b, and 3b, so as to confine the light within the detector.

In the example of FIG. 3, a laser pulse at a wavelength within the absorbing range of PI layer 2 (e.g. 355 nm) is delivered through SMF 1 and is absorbed by PI layer 2. The pulse absorption generates ultrasound waves by the thermoelastic mechanism. A second laser (CW at 1550 nm) is delivered through SMF 1 and is used to probe the etalon structure of layers 3a, 4a, 4b, 3b. The reflection of the CW laser is measured by the PD. The PD output is converted from current to voltage and then sampled by the A/D converter. The digital signal corresponds to the acoustic pressure at the active area of the device.

The construction of FIG. 3 may similarly be applied to the device of FIG. 2. In this case, PI layer 2, disposed between layers 3a, 3b, may be formed to have two parts having different refraction indices so as to extend the fiber cladding and core into the etalon structure.

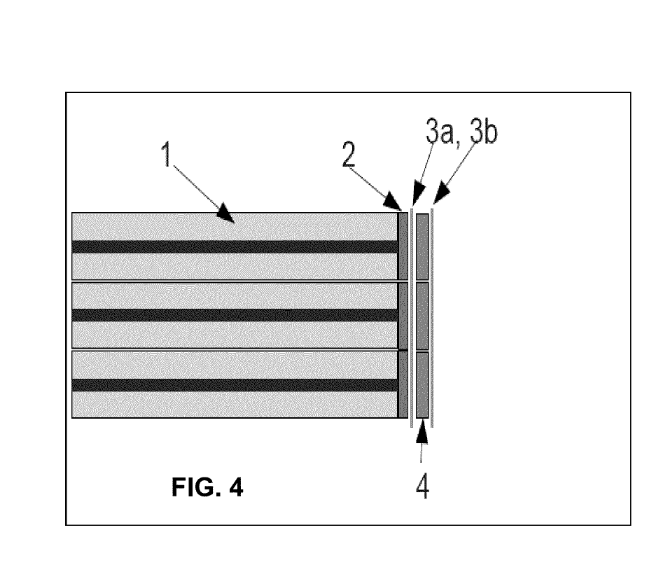

FIG. 4 is a block diagram illustrating an example optical ultrasound transducer, in accordance with one or more aspects of the present disclosure. As shown, a bundle of single mode fibers or multimode fibers (i.e., a group of two or more of SMF 1) are each coated with a PI layer 2. In the example of FIG. 4, PI layer 2 may typically be 1-2 μm in width. Each of SMF 1 may also be coated with a first reflecting surface, layer 3a, a layer of transparent polymer, layer 4, and a second reflecting surface, layer 3b. In this way, an ultrasound transducer having multiple imaging elements may be formed from the example embodiment of FIG. 1. Although not shown, the example embodiment of FIG. 2 may be arranged in a similar manner to form an ultrasound transducer of multiple elements.

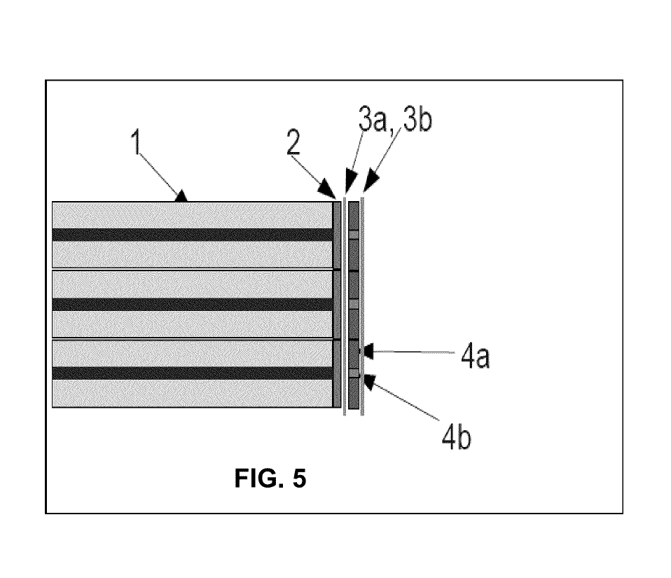

FIG. 5 is a block diagram illustrating an example optical ultrasound transducer, in accordance with one or more aspects of the present disclosure. In this example, each fiber in a bundle of single mode fibers or multimode fibers 1 is coated with PI layer 2, typically 1-2 μm in width, and a first reflecting surface, layer 3a. A layer of transparent polymer, layer 4b, is then coated on the surface of layer 3a. Layer is then patterned to remove material not in front of the core of each of SMF 1. A second polymer layer, layer 4a, is then applied to fill the space. A second reflecting surface, layer 3b, is then coated on. The optical refraction index of layer 4b is higher than the refraction index of layer 4a, thereby extending each of SMF or MMF 1 into the detector. In this way, an ultrasound transducer having multiple imaging elements may be formed from the example embodiment of FIG. 3.

In this way, as shown in FIGS. 1-5, the UV pulse for HFUS generation may be integrated into the optical assembly used for etalon detection. These pulses are directed through the same lens used to focus the NIR beam, which allows the transmitting and sensing elements to be precisely in the same location. The resonance wavelength may be prerecorded at each detector site so as to compensate for the change in etalon thickness encountered during beam scanning This facilitates acquisition of the maximal signal available at each detection site. Coupling the UV light to a multi-mode optical fiber provides a fiber optic HFUS imager. In some examples, an optical ultrasound transducer, in accordance with one or more aspects of the present disclosure, may be used in combination with other optical imaging methods. For instance, an optical ultrasound transducer as exampled in one of FIGS. 1-5 may be used with photoacoustic imaging, optical imaging (i.e., endoscopy), fluorescence imaging, optical coherence tomography (OCT), or other optical imaging methods. Such a combination is possible due to the limited absorption ranges of the optical ultrasound transducer. That is, the optical ultrasound transducer as described in the present disclosure may be used in connection with a third light source or more light sources. The third light source may provide light at a third wavelength, within the transmission range of PI layer 2, used for other optical imaging methods. In some examples, the third light source may be white light, or other broadband illumination sources.

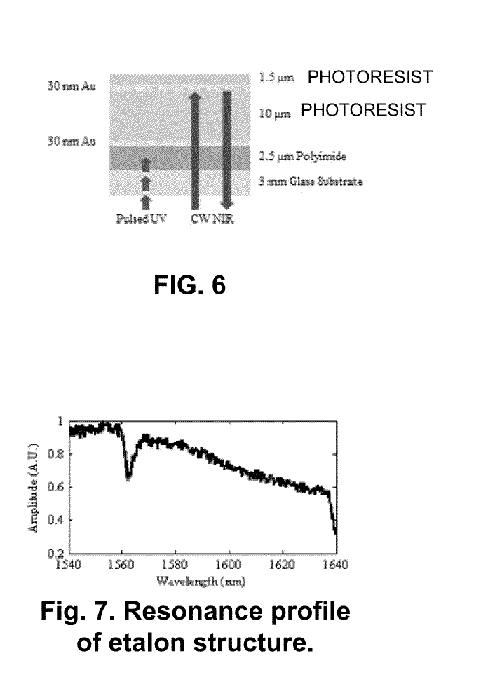

FIG. 6 is a block diagram illustrating an example layered microstructure and operating principle of an all-optical, thin-film, high-frequency ultrasound transducer, in accordance with one or more aspects of the present disclosure. In this example, a polyimide adhesion promoter is spin-coated onto a glass substrate having a 25 mm diameter and 3 mm thickness. A layer of polyimide precursor is spin-coated onto the substrate. The polyimide precursor has a thickness of approximately 2.5 μm. The sample may be heated to 250° C. at a rate of 10° C./min and then cured (e.g., for 90 minutes) in nitrogen. After gradually cooling to room temperature, a first etalon mirror, e.g., a 3/30/3 nm Ti/Au/Ti film, is deposited on top of the polyimide film using electron-beam evaporation. A 10 μm layer of photoresist is then spin-coated, cured, and exposed to UV light for cross-linkage. A second etalon mirror, identical to the first, is then deposited. Additionally, 1.5 μm of photoresist may be added to provide a layer of protection. In operation, pulsed UV is absorbed by the polyimide layer which launches an acoustic wave. The etalon, which operates at NIR wavelengths, detects the echo.

In operation, the example device of FIG. 6 may produce an optically-generated acoustic pulse having an amplitude of 4.3 MPa and a −3 dB bandwidth of 29 MHz centered at 27 MHz. The etalon sensor may achieve a Noise-equivalent Pressure of 1.3 Pa/\sqrt{Hz}. When used in pulse-echo mode, the −6 dB upper cutoff frequency of the device's transmit/receive response may reach 47 MHz. A 1-D synthetic aperture can be created, and imaging results may reach an upper limit of 100 μm and 40 μm on the lateral and axial resolution, respectively.

With the incorporation of fiber optics and 2-D beam scanning, aspects of the present disclosure may be applied, for example, in endoscopic and intravascular ultrasound. For instance, a transmitting film may be used that is (1) easy to fabricate, (2) of a high damage threshold, and (3) sufficiently transparent to wavelengths used for etalon sensing. This would allow the sensing and transmitting elements to be in the same location and would allow the transmitting film to be placed underneath the etalon. In one example, a polyimide precursor PI-2555, a material known for its resistance to high temperatures and characteristic optical absorption in the UV spectrum, may be used for PI layer 2 in FIGS. 1-5.

FIG. 7 is a graphical diagram showing an example resonance profile of the etalon sensor of FIG. 6 when optically tested, in accordance with one or more aspects of the present disclosure. A fiber-connectorized output of a NIR wavelength-tunable CW laser may be routed to a fiber optic collimator with a polarization maintaining fiber. An optical circulator can be implemented using a polarizing beam-splitter and quarter-wave plate. Following the wave plate, the 3 mW beam may be focused onto the etalon with a spot size of 26 μm in diameter. The beam may then be reflected back through the circulator and detected using a DC IR power meter (e.g., a Thorlabs PM100). The NIR laser wavelength can then be swept throughout its tunable range (1508-1640 nm), and the analog output of the power meter may be digitally acquired at each wavelength. The Q-factor and Finesse may achieve 453 and 23, respectively.

The acoustic performance of the sensing and transmitting elements were next verified independently. The IR power meter may be replaced with a high-speed InGaAs photodetector. After tuning the wavelength of the CW NIR beam for maximum sensitivity, a 25 MHz ultrasound probe may be driven by a Pulsar/Receiver unit and focused onto the etalon structure in water.

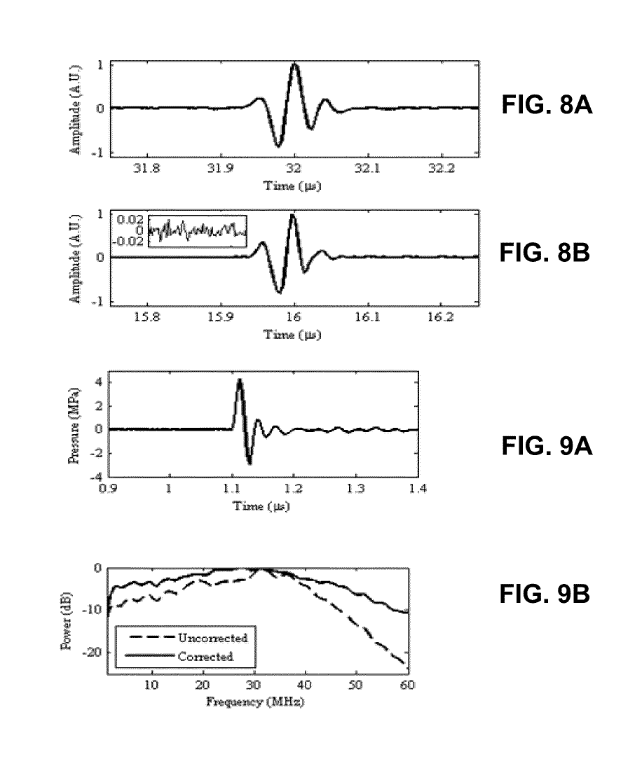

FIGS. 8A and 8B are graphical diagrams showing example pulse-echoes from an optical ultrasound transducer in accordance with one or more aspects of the present disclosure. FIG. 8A shows a pulse-echo of a 25 MHz transducer using an etalon structure as a reflecting target. FIG. 8B shows etalon detection of a 25 MHz pulse with an inset zoomed in on noise preceding the pulse. In FIG. 8A can be found the pulse-echo off of the etalon as detected by the probe, bandpass filtered from 2.5 to 50 MHz. Signals may be sampled at 250 MHz with an 8-bit digitizer (e.g., NI PXI-5114). FIG. 8B shows the same pulse as may be detected by the etalon, amplified by 30 dB and band-pass filtered from 2.5 to 50 MHz. The maximum pressure generated by the probe may be measured to be 1.13 MPa using a calibrated hydrophone (e.g., Onda HGL-0085). Based on a maximum amplitude (31.2 mV), and the RMS value of the noise prior to the main pulse (0.26 mV), the Noise-equivalent Pressure (NEP) would then be 8.9 kPa or 1.3 Pa/\sqrt{Hz}. Dividing the square of the maximum pressure by the square of the NEP results in a signal-to-noise ratio of 127.

A 5 ns 4 mJ 355 nm pulse from a ND:YAG laser may be directed towards the device at an incident angle of roughly 60 degrees. The area of illumination may be elliptical with a major diameter of 3.3 mm and minor diameter of 2.3 mm, yielding a fluence of 67 mJ/cm2. The bandwidth and amplitude of the acoustic signal generated by the polyimide film can then be measured using the hydrophone from a distance of 1.6 mm.

FIGS. 9A and 9B are graphical diagrams showing a recorded waveform and associated frequency response of an optical ultrasound transducer, in accordance with one or more aspects of the present disclosure. In the examples of FIGS. 9A, 9B, the waveform may be averaged 16 times. In particular, FIG. 9A shows the signal generated by polyimide film with 355 nm pulse as measured using a calibrated hydrophone, averaged 16 times. FIG. 9b shows uncorrected and corrected power spectrums of the waveform. The power spectrum of the waveform may be corrected by dividing it by the square of the sensitivity spectrum (original units of V/Pa) provided with the calibrated hydrophone (up to 60 MHz). Based on the corrected spectrum, the center frequency and peak response occurred at 27 MHz with a −3 dB bandwidth of 29 MHz. The mean sensitivity across the −3 dB bandwidth (56 nV/Pa) may be used to convert the vertical scale of the waveform from volts to pascal, thereby indicating a maximum generated pressure of 4.3 MPa.

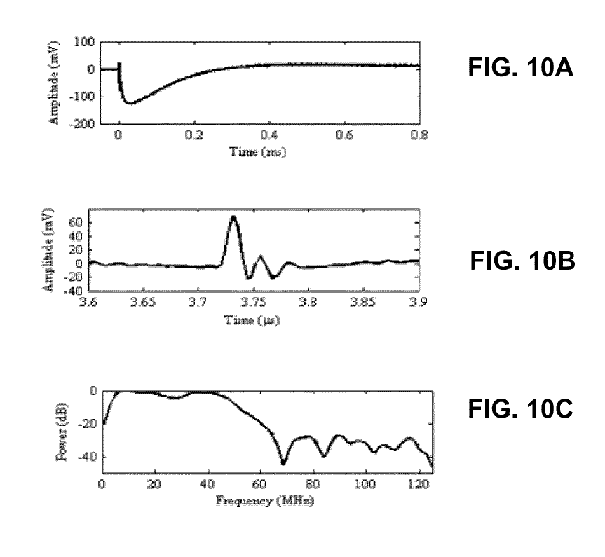

FIGS. 10A-10C are graphical diagrams showing etalon detection of a polyimide pulse-echo in an optical ultrasound transducer, in accordance with one or more aspects of the present disclosure. The example device of FIG. 6 may be configured to operate in transmit/receive mode. In order to do so, the hydrophone may first be removed, and the signal detected by the etalon with no target may then be recorded. In particular, FIG. 10A shows a heat signal generated by a UV pulse with no target present. FIG. 10A shows the signal created by the heating of the etalon with the UV pulse. Its time course is on the order of 100 μs. Thus it is separable from the signals of interest by use of filtering. A glass slide may be placed 2.7 mm away from the device, and the pulse emitted by the polyimide film may be reflected off of the slide and detected by the etalon. FIG. 10B shows pulse-echo off of a glass-slide, high-pass filtered at 4 MHz. FIG. 10C illustrates the power spectrum of the pulse-echo. A high-pass filter with a cutoff frequency of 4 MHz may be used to filter out the heat signal. FIG. 10C shows, as one example, the upper cutoff frequency of the transmit/receive response falling below −6 dB at 47 MHz.

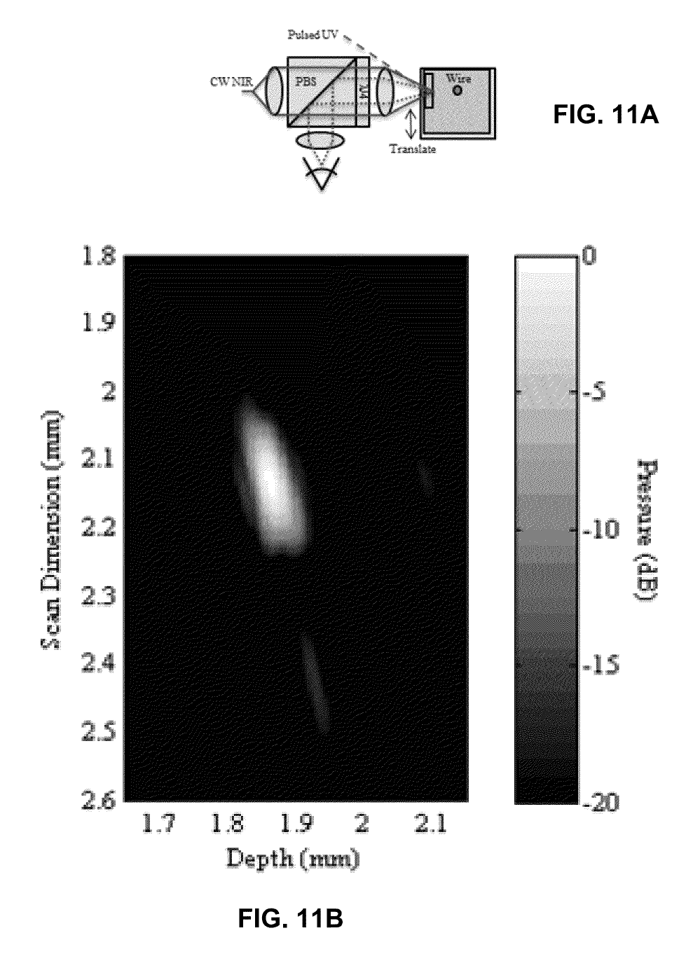

FIG. 11A and 11B are a block diagram and associated graphical diagram illustrating an example 1-D synthetic aperture scanning system, in accordance with one or more aspects of the present disclosure. FIG. 11A illustrates an experimental setup for the 1-D synthetic aperture scanning The imaging capabilities of the all-optical transducer may be tested by placing a wire of 250 μm in diameter approximately 2 mm away from the device. The optical assembly can then be translated perpendicular to the wire's axis in order to create a 1-D synthetic imaging aperture. The IR beam may be scanned across a 1.4 mm line using a step size of 10 μm resulting in a 142 sensor array. Signals may again be sampled at 250 MHz, and 16 waveforms acquired and averaged at each location. After band-pass filtering the signals from 20 to 50 MHz, image reconstruction may be performed using a basic beam-forming algorithm. FIG. 11B shows the result, which indicates an upper limit on the −6 dB resolution, approximately 40 μm in the axial dimension and 100 μm in the lateral dimension. In general, even better image quality and higher resolution may be achieved by upgrading to a 2-D aperture.

As such, the constructed model demonstrated the functionality of an all-optical high-frequency ultrasound transducer. An optically-absorbing polyimide thin-film generated a 4.3 MPa signal, and the etalon sensor exhibited an NEP of 1.3 Pa/\sqrt{Hz}. The −6 dB transmit/receive response reached 47 MHz, and lateral and axial resolutions of 100 μm and 40 μm, respectively, were achieved using a 1-D synthetic aperture.

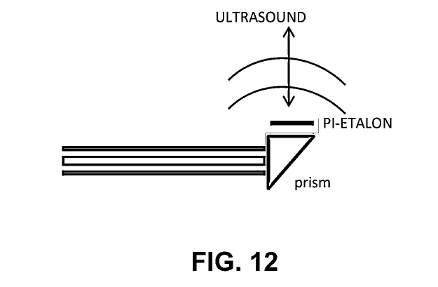

FIG. 12 is a block diagram illustrating an example configuration of an optical ultrasound transducer, in accordance with one or more aspects of the present disclosure. The optical ultrasound transducer, as described, may be combined with other optical components to achieve line of sight in different directions. As seen in the example of FIG. 12, a prism may be placed between the SMF (e.g., SMF 1 of FIGS. 1-5) and the etalon structure, in order to achieve a right-angle light of sight. In other examples, the fiber itself (e.g., SMF 1 of FIGS. 1-5) may be polished in differing degrees (e.g., a 45 degree angle), in order to achieve various viewing angles. In this way, the optical ultrasound transducer may be utilized in implementations involving side-viewing imaging devices.

The device described herein may be incorporated into a forward-viewing IVUS imager for evaluating Chronic Total Occlusion (CTO). The forward-viewing IVUS may operate at frequencies beyond 50 MHz, for example, and provide a sufficiently high resolution (30-200 μm) while retaining an adequate penetration depth (2-10 mm). While a few groups have successfully developed CMUT-based forward-viewing IVUS, a frequency response above 35 MHz has yet to be demonstrated. The results described herein indicate that bandwidth of the device could easily be increased beyond 50 MHz by reducing the 10 μm photoresist layer to 5 μm, for example. In addition, the techniques described herein may allow simple and low-cost fabrication of a transmit/receive etalon relative to CMUT arrays. Moreover, the device described herein need not require electrical connections because of its use of light delivery. The coupling of the device to an optical fiber bundle provides a flexible, compact, and robust design, which may have particular applicability for IVUS.

Various embodiments of the invention have been described. These and other embodiments are within the scope of the following claims.

1. An optical ultrasound transducer comprising:
an optical fiber;
a polymer layer formed on the optical fiber to receive light from the optical fiber;
wherein the polymer layer absorbs light of a first wavelength and generates an acoustic tone in response to the light of the first wavelength, and wherein the polymer layer is substantially transparent to light of a second wavelength; and
an optical detector formed on the polymer layer, wherein the optical detector comprises an etalon structure having a first mirror layer and a second mirror layer separated by a compressible layer, wherein the compressible layer resonates in response to the light of the second wavelength passing through the polymer layer and is compressible in response to acoustic pressure from echoes of the acoustic tone.

2. The optical ultrasound transducer of claim 1, wherein the polymer layer absorbs ultra-violet (UV) light and generates the acoustic tone in response to the UV light, and wherein the compressible polymer layer is transparent to near-infrared light.

3. The optical ultrasound transducer of claim 1, wherein the polymer layer comprises a polyimide (PI) polymer having an absorption range of approximately 200 nm to 400 nm and a transmission range of approximately 600 nm to 2000 nm.

4. The optical ultrasound transducer of claim 1, wherein the compressible layer comprises an inner material and an outer material, wherein the inner material is aligned with a core of the optical fiber and has a higher refraction index than the outer material.

5. The optical ultrasound transducer of claim 1, wherein the optical fiber comprises a single mode optical fiber.

6. The optical ultrasound transducer of claim 1, wherein the optical fiber comprises a multimode optical fiber.

7. The optical ultrasound transducer of claim 1, further comprising a bundle of optical fibers, each having a polymer and an optical detector formed on an end of the optical fiber.

8. The optical ultrasound transducer of claim 1, wherein the compressible layer resonates in response to the light of a third wavelength passing through the polymer layer, and illuminates the environment.

9. An optical ultrasound transducer comprising:
an optical fiber; and
an optical detector to receive light from the optical fiber, wherein the optical detector comprises an etalon structure having a first mirror layer and a second mirror layer separated by a compressible layer,
wherein the compressible layer comprises a polymer that generates an acoustic tone in response to the light of the first wavelength, and
wherein the etalon structure resonates in response to light of a second wavelength and is compressible in response to acoustic pressure from echoes of the acoustic tone.

10. The optical ultrasound transducer of claim 9,
wherein the first mirror layer is a wavelength-selective mirror that transmits ultra-violet (UV) light to the polymer and reflects near infra-red light, and
wherein the polymer absorbs the UV light and generates the acoustic tone in response to the UV light, and wherein the polymer is transparent to near-infrared light.

11. The optical ultrasound transducer of claim 9, wherein the polymer comprises a polyimide (PI) polymer having an absorption range of approximately 200 nm to 400 nm and a transmission range of approximately 600 nm to 2000 nm.

12. The optical ultrasound transducer of claim 9, wherein the compressible layer comprises an outer material formed around the polymer, wherein the polymer is aligned with a core of the optical fiber and has a higher refraction index than the outer material.

13. The optical ultrasound transducer of claim 9, wherein the optical fiber comprises a single mode optical fiber.

14. The optical ultrasound transducer of claim 9, wherein the optical fiber comprises a multimode optical fiber.

15. The optical ultrasound transducer of claim 9, further comprising a bundle of optical fibers, each having a polymer layer and an optical detector formed on an end of the optical fiber.

16. The optical ultrasound transducer of claim 9, wherein the polymer layer and the optical detector have a width of 10 microns or less.

17. The optical ultrasound transducer of claim 9, further comprising a prism, wherein the prism receives light from the optical fiber at a first angle, and provides the light to the optical detector at a second angle, the first and second angles being different from one another.

18. A method of constructing an optical ultrasound transducer comprising:
coating a polymer layer on an end of an optical fiber, wherein the polymer layer has an optical absorption range in which the polymer layer generates an acoustic tone in response to light of a first wavelength and has an optical transmission range in which the polymer layer is substantially transparent to light of a second wavelength; and
forming an etalon structure on the polymer layer, wherein the etalon structure is formed as a first mirror layer and a second mirror layer separated by a compressible layer, wherein the compressible layer resonates in response to the light of the second wavelength and is compressible in response to acoustic pressure from echoes of the acoustic tone.

19. The optical ultrasound transducer of claim 18, wherein forming the etalon structure comprises forming the compressible layer as an inner material aligned with a core of the optical fiber and an outer material around the inner material, wherein the inner material has a higher refraction index than the outer material.

20. The optical ultrasound transducer of claim 18, further comprising polishing the end of the optical fiber at an angle other than perpendicular to a core of the optical fiber.
